(19) United States
(12) Patent Application Publication
(10) Pub. No.: US 2019/0264246 A1
(43) Pub. Date: Aug. 29, 2019

(54) PROPAGATION OF YEAST USING CELLULOSE AS A CARBON SOURCE

(71) Applicant: DOMTAR PAPER COMPANY, LLC, FORT MILL, SC (US)

(72) Inventor: Neelakantam V. Narendranath, Fort Mill, SC (US)

(21) Appl. No.: 16/282,852

(22) Filed: Feb. 22, 2019

Related U.S. Application Data
(60) Provisional application No. 62/634,625, filed on Feb. 23, 2018.

(51) Int. Cl. C12P 19/02; C12P 7/10; C12N 1/16

(52) U.S. Cl. CPC C12P 19/02; C12P 7/10; C12N 1/16; C12Y 302/01021; C12Y 302/01091; C12Y 302/01032; C12Y 302/01004

(57) ABSTRACT

Methods for propagation of yeast using cellulosic material as a carbon source are disclosed. A propagation medium is provided by combining a nutrient source, a cellulosic material to be used as a carbon source, enzymes that hydrolyze the cellulosic material into monosaccharides that can be taken up and metabolized by yeast, and a first cell mass of yeast. The propagation medium is incubated in a temperature range that allows the enzymes to break down the cellulosic material into monosaccharides at a rate that leads to production of a second cell mass of yeast, while keeping ethanol production low enough that the concentration of ethanol does not exceed 0.1 grams per liter of propagation medium.

### CROSS-REFERENCE TO RELATED APPLICATIONS

This application claims priority to and the benefit of U.S. Provisional Application No. 62/634,625, filed Feb. 23, 2018. The contents of the referenced patent application is incorporated into the present application by reference.

### FIELD OF INVENTION

The present invention relates generally to systems and methods for propagating yeast using cellulosic material as a carbon source. More particularly, one or more cellulolytic enzymes are used to break down the cellulosic material into one or more monosaccharides at a rate conducive to supporting growth of yeast biomass with minimal or no production of ethanol.

### DESCRIPTION OF RELATED ART

The yeast Saccharomyces cerevisiae is propagated by yeast manufacturers predominantly using molasses as carbon source. However, the amount of molasses that can be used, and hence the growth rate of yeast biomass, is limited by the Crabtree effect. The Crabtree effect is a phenomenon in which yeast ferment glucose to ethanol when the sugar level is greater than 5 g/L, even under highly aerobic conditions. Thus, growing yeast on molasses can lead to unwanted ethanol production and consequently a lower rate of yeast biomass production. Since the yeast manufacturers are more interested in producing yeast biomass then ethanol, molasses concentrations during yeast propagation are kept below a level that would induce the Crabtree effect. This requires using a fed-batch process in which the sugar is slowly fed to the reactor to avoid accumulating an excess of glucose in the reactor. This allows the yeast to be in respiratory growth mode to efficiently increase biomass, rather than in a fermentative mode making ethanol. In the fed-batch yeast production process, the off-gas is constantly monitored for ethanol levels, and this information is used to control the flow of sugar/molasses into the reactor. This fed-batch process can be complicated and expensive relative to a simple batch process.

Cellulosic biomass is an abundant source of polysaccharides that can potentially be used as a carbon source for yeast propagation. However, current methods of growing yeast using cellulose do not efficiently produce yeast biomass in a batch process, and also end up producing substantial amounts of ethanol. Known methods of growing yeast on cellulosic biomass typically are not simple batch methods in which conversion of cellulose poly- and oligosaccharides into monosaccharides take place at the same time, in the same space, and under the same conditions in which yeast can propagate without substantial production of ethanol. Instead, they typically involve a separate step performed at relatively high temperatures, for example 50° C., in which enzymes break down cellulose into monosaccharides. Only after production of the monosaccharides are they provided to a yeast culture. These processes lack the efficiency of a batch process in which the enzymes break down the cellulose under the same conditions that the yeast propagate, and the fermentative production of ethanol in these known processes prevents efficient production of yeast biomass.

Methods of producing yeast in a simple batch process using complex carbohydrates as the carbon source have been previously disclosed. For example, U.S. Pat. No. 9,034,631 to Narendranath & Lewis discloses growing yeast in a batch process using starch as a carbon source. The yeast and starch are incubated with amylolytic enzymes that break down the polysaccharides and oligosaccharides of the starch into monosaccharides that the yeast can use to grow. However, these methods cannot be readily applied to propagation of yeast using cellulosic biomass as a carbon source because the amylolytic enzymes that break down starch would not readily break down cellulose into usable monosaccharides.

### SUMMARY

This disclosure includes a solution to the inefficiency of yeast biomass growth due to unwanted ethanol production in batch propagation processes. The solution involves providing cellulosic material as a carbon source for yeast propagation in a batch propagation process along with enzymes that can hydrolyze the cellulosic material into useable monosaccharides. These cellulolytic enzymes require different sets of conditions for efficient production of monosaccharides at a rate conducive to yeast biomass production without substantial ethanol production. Maintaining these conditions—such as enzyme concentration, temperature, and pH—at levels as described herein leads to production of monosaccharides at a rate that is high enough to support efficient yeast biomass growth and low enough to avoid ethanol production resulting from the Crabtree effect.

In one embodiment, a method of propagating yeast is disclosed. The method comprises (a) combining a propagation medium comprising a nutrient source, a carbon source comprising cellulosic material, one or more enzymes that can convert at least a portion of the cellulosic material into one or more monosaccharides, and a first cell mass of yeast; (b) enzymatically converting at least a portion of the cellulosic material in the propagation medium into one or more monosaccharides; and (c) incubating the propagation medium for sufficient time period to form a second cell mass of yeast that has a higher dry cell weight than the first cell mass of yeast; wherein no more than 0.1 g/L of ethanol is produced in the propagation medium by the yeast during step (c). In some embodiments substantially no ethanol is produced by the yeast during step (c). Substantially no ethanol is produced if no more than trace amounts of ethanol can be detected in the propagation medium after the incubation is complete. In some embodiments, step(b) and step (c) occur simultaneously for at least a period of time. In some embodiments, the pH of the propagation medium is maintained in a range between about 4 and 6 or between about 5.0 and 5.2.

In some embodiments, the first cell mass of yeast has a dry cell weight of about 0.05 to 0.50 g per liter of propagation medium. Incubation during step (c) causes the first cell mass of yeast to propagate and produce a second cell mass of yeast. The second cell mass of yeast may be several fold larger, as measured by either dry cell weight of yeast or yeast cell number. In some embodiments, the second cell mass of yeast has a dry cell weight of approximately 5 to 15 g per liter of propagation medium. In some embodiments, the second cell mass has a dry cell weight approximately 25 to 100 times that of the first cell mass of yeast. In some embodiments, the yeast are Saccharomyces cerevisiae, which may be wild-type or may be engineered to have certain genetic modifications. The genetic modifications may, for example, expand the range of monosaccharides that the yeast can metabolize and use as a carbon source to support propagation and biomass growth. In some embodiments, the propagation medium is incubated for 12 to 24 hours. The propagation medium may be maintained at a temperature in the range from about 25 to 37° C. or in the range from about 30 to 32° C.

The cellulosic material combined with the propagation medium may comprise any material that includes cellulose and may be derived from a variety of sources, including wood pulp, corn stover, wheat straw, switch grass, and the like. In some embodiments, the cellulosic material comprises lignocellulosic material that includes both lignin and cellulose. In some embodiments, the cellulosic material has been pretreated. In some embodiments, the cellulosic material comprises cellulose derived from pulp generated by the kraft or sulfite pulping process.

The enzymes combined with the propagation medium serve to break hydrolyze the polysaccharides and oligosaccharides in the cellulosic material into monosaccharides that can be taken up and metabolized by the yeast. In some embodiments, the one or more enzymes comprise one or more of an endoglucanase, a cellobiohydrolase, and a beta-glucosidase. The enzymes may be added in the form of an enzyme cocktail. The enzymes are added in amounts that are effective to produce monosaccharides at a rate conducive to yeast biomass growth without exceeding a monosaccharide concentration that would induce ethanol production. “Effective” and any variations thereof means adequate to accomplish a desired, expected, or intended result.

In some embodiments, the one or more enzymes are present in the propagation medium at a total enzyme concentration of about 0.25 to 0.35 grams of enzyme per gram of cellulosic material or about 0.02 to 0.7 grams of enzyme per gram of cellulosic material. The concentrations of individual enzymes can be adjusted and optimized to provide for production of monosaccharides at a rate conducive to yeast biomass growth without exceeding a monosaccharide concentration that would induce ethanol production. The temperature and pH of the propagation medium may also affect the rate of hydrolysis and can be adjusted to provide for monosaccharide production at a desired rate.

The terms “a” and “an” are defined as one or more unless this disclosure explicitly requires otherwise.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “substantially,” “about,” and “approximately” are defined as largely but not necessarily wholly what is specified—and includes what is specified; e.g., substantially 90 degrees includes 90 degrees and substantially parallel includes parallel—as understood by a person of ordinary skill in the art. In any disclosed embodiment, the terms “about” and “approximately” may be substituted with “within [a percentage] of” what is specified, where the percentage includes 0.1, 1, 5, and 10 percent.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. For example, the methods of introducing substances into cells disclosed herein can “comprise,” “consist essentially of,” or “consist of” particular components, compositions, ingredients, etc. disclosed throughout the specification.

Other objects, features and advantages of the present invention will become apparent from the following FIGURES and detailed description. It should be understood, however, that the figures and detailed description, while indicating specific embodiments of the invention, are given by way of illustration only and are not meant to be limiting. Additionally, it is contemplated that changes and modifications within the spirit and scope of the invention will become apparent to those skilled in the art from this detailed description.

The feature or features of one embodiment may be applied to other embodiments, even though not described or illustrated, unless expressly prohibited by this disclosure or the nature of the embodiments. Some details associated with the embodiments described above and others are described below.

### BRIEF DESCRIPTION OF THE DRAWINGS

FIG. 1 shows an illustration of an embodiment of a yeast propagation system in which yeast biomass can be produced efficiently with substantially no production of ethanol.

### DETAILED DESCRIPTION OF ILLUSTRATIVE EMBODIMENTS

The yeast methods disclosed herein efficiently produce yeast biomass using cellulosic material as a carbon source. The yeast biomass is produced in a propagation medium, which generally includes at least a nutrient source, a carbon source, and water. The conditions of the propagation medium, such as temperature, pH, aeration, and enzyme concentrations affect the efficiency at which the yeast propagate and the amount of ethanol produced. These and other aspects of the disclosed method will be described in greater detail below.

### A. Cellulosic Material

As used herein, the term “cellulosic material” means any material containing cellulose. Cellulosic material is highly abundant and can be found, for example in stems, leaves, hulls, husks, cobs, branches, and wood of plants. Cellulosic material derived from plants may include a combination of polysaccharides including, for example, cellulose, hemicellulose, and pectin. In some embodiments, the cellulosic material is lignocellulosic material that comprises both lignin and cellulose.

Cellulosic material can be derived from agricultural residue, herbaceous material (including energy crops), municipal solid waste, pulp and paper mill residue, waste paper, and wood (including forestry residue). The cellulosic material may include without limitation any of the following: arundo, bagasse, bamboo, corn cob, corn fiber, corn stover, miscanthus, orange peel, rice straw, switchgrass, wheat straw, aspen, eucalyptus, fir, pine, poplar, spruce, willow, algal cellulose, bacterial cellulose, and microcrystalline cellulose.

The cellulosic material may be subjected to particle size reduction, sieving, pre-soaking, wetting, washing, and/or conditioning before being added to a propagation medium for use in the methods described herein. The cellulosic material may also be subjected to other pretreatments, including without limitation any of the following, either alone or in combination: steam pretreatment, acid pretreatment, hot water pretreatment, alkaline pretreatment, lime pretreatment, biological pretreatment, ammonia percolation, ultrasound pretreatment, electroporation pretreatment, microwave pretreatment, ozone pretreatment, ionic liquid pretreatment, and mechanical pretreatment. Such pretreatments may promote the separation and/or release of cellulose, hemicellulose, and/or lignin. Pretreatment may convert crystalline cellulose to amorphous cellulose.

In some aspects, the pretreatment makes the cellulosic material more susceptible to efficient enzymatic conversion to monosaccharides. This can be accomplished by, for example, reducing the length of glucan chains and/or removing substances that inhibit enzymatic hydrolysis of the cellulosic material or that inhibit yeast growth.

### B. Enzymes

Methods described herein employ enzymes to convert polysaccharides and oligosaccharides in cellulosic material to monosaccharides (e.g., glucose) that can be taken up and metabolized by yeast. The enzymes may be cellulolytic or hemicellulolytic enzymes (i.e., cellulases and/or hemicellulases) and may include, for example, one or more of the following types of enzymes, alone or in combination: endoglucanases, cellobiohydrolases, beta-glucosidases, glucoside hydrolases, acetylmannan esterases, acetylxylan esterases, arabinases, arabinofuranosidases, coumaric acid esterases, feruloyl esterases, galactosidases, glucuronidases, glucronoyl esterases, mannases, mannosidases, oxidoreductases, xylanases, xylosidases, and beta-xylosidases. The enzymes may comprise one or more of cellobiohydrolase I, cellobiohydrolase II, endo-1,4-β-glucanase, exo-1,4-β-glucanase, and 1,4-β-glucosidase.

In one aspect, the enzymes comprise a commercial enzyme preparation. Examples of commercial enzyme preparations suitable for use in the disclosed methods include, for example, CELLIC® CTec (Novozymes A/S), CELLIC® CTec2 (Novozymes A/S), CELLIC® CTec3 (Novozymes A/S), CELLUCLAST® (Novozymes A/S), NOVOZYM™ 188 (Novozymes A/S), SPEZYME™ CP (Genencor Int.), ACCELLERASE™ TRIO (DuPont), FILTRASE® NL (DSM); METHAPLUS® S/L 100 (DSM), ROHAMENT™ 7069 W (Rohm GmbH), or ALTERNAFUEL® CMAX3™ (Dyadic International, Inc.).

In some embodiments, the cellulolytic enzymes are present in the propagation medium at a total enzyme concentration of about 0.02 to 0.7 grams of enzyme per gram of cellulosic material in the propagation medium. In some embodiments, the total enzyme concentration is about 0.25 to 0.35 grams of enzyme per gram of cellulosic material in the propagation medium. In some embodiments, the total enzyme concentration is at least about, at most about, or is about 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.90 grams of enzyme per gram of cellulosic material in the propagation medium, or is between any two of these values. In some embodiments, 87.5 to 1225 biomass hydrolysis units (“BHU(2)”) of enzymes are present in the propagation medium. In some embodiments, 430 to 615 BHU(2) of enzymes are present in the propagation medium. In some embodiments, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, or 1250 BHU(2) of enzymes are present in the propagation medium, or between any two of these values.

In some embodiments, the concentration of an individual enzyme is at least about, at most about, or is about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.90 grams of enzyme per gram of cellulosic material in the propagation medium, or is between any two of these values. These concentration values can be the concentration of any one of an endoglucanase, cellobiohydrolase, beta-glucosidases, xylanase, and/or beta-xylosidase in the propagation medium. These concentration values can be the concentration of cellobiohydrolase I, cellobiohydrolase II, endo-1,4-β-glucanase, exo-1,4β-glucanase, and/or 1,4-β-glucosidase, or other enzymes disclosed herein in the propagation medium. Each of these enzymes may be present at one of the above concentration values in any combination with one or more of any other cellulolytic enzyme.

An appropriate amount of enzymes to be used in the propagation medium can be determined by monitoring the rate of production of monosaccharides (e.g., glucose) by the enzymes under the particular propagation conditions. In some embodiments, the enzyme concentration at a given temperature is chosen such that the concentration of glucose in the propagation medium remains below a level that would induce the Crabtree effect and cause production of ethanol. In some embodiments, the enzymes are present in the propagation medium at an amount that results in a concentration of glucose in the propagation medium to be less than or equal to about 0.5, 1.0, 1.5, 2.0, 2.5, 3.0, 3.5, 4.0, 4.5, or 5.0 g/L, or between any two of these values. In some embodiments, the amount of enzymes may be increased as the yeast biomass increases to provide sufficient production of glucose to sustain efficient yeast biomass production.

In some embodiments, the only cellulolytic enzymes present in the propagation medium are enzymes that are not produced by yeast cells in the first cell mass of yeast—that is, the cell mass of yeast that is inoculated into the propagation medium. In such embodiments, the enzymes may be added to the propagation medium separately from the yeast. In some embodiments, at least a portion of the enzymes are produced by the yeast themselves. Enzymes that can convert cellulosic material into monosaccharides may be produced by genetically modified yeast. In some embodiments, the enzymes may be a combination of separately added enzymes and enzymes produced by the yeast. In any case, the total amount of enzymes in the propagation medium (i.e., the enzymes added to the propagation medium and the enzymes produced by the yeast in the propagation medium) may be within the concentration and activity ranges disclosed above.

### C. Yeast

The disclosed methods can be used for production of any type of yeast used for ethanol production, baking, wine making, distilling, and animal feed. The yeast produced by the propagation methods disclosed herein may be, for example, strains of Saccharomyces, Candida, Torula, and Kluyveromyces genera. In some embodiments, the yeast comprise Saccharomyces cerevisiae, Candida sonorensis, Candida utilis, or Kluyveromyces marxianus. The yeast may be wild-type or may have genetic modifications. Examples of genetic modifications include, for example, causing yeast to express enzymes that enable them to metabolize five-carbon sugars such as xylose. Yeast may also be modified to express cellulolytic enzymes.

In some embodiments, the yeast are inoculated into the propagation medium at a concentration of 0.01 to 10 g of dry cell weight per liter of the propagation medium. In some embodiments, the concentration is between about 0.02 and 5 g of dry cell weight per liter of the propagation medium. The yeast inoculated into the propagation medium is referred to herein as a first cell mass of yeast. In some embodiments, the first cell mass of yeast has a concentration of at least about, at most about, or about 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 g dry cell weight per liter of propagation medium, or between any two of these values.

Propagating the yeast according to the methods described herein causes the yeast biomass in the propagation medium to increase efficiently. In some embodiments, incubating the propagation medium for a time between 12 and 24 hours causes the yeast biomass to increase by at least 100-fold with no or minimal production of ethanol (e.g., less than 0.1 g/L of ethanol). In some embodiments, the increase in yeast biomass is at least about 10, 15, 20, 25, 30, 35, 40, 45, 50, 55, 60, 65, 70, 75, 80, 85, 90, 95, 100, 105, 110, 115, 120, 125, 130, 135, 140, 145, or 150-fold or is between any two of these values. The yeast biomass produced during the incubation of the propagation medium is referred to herein as the second cell mass of yeast. In some embodiments, after incubating the propagation medium, the second cell mass of yeast has a concentration of at least about, at most about, or about 5, 10, 15, 20, 25, or 30 g dry cell weight per liter of propagation medium, or between any two of these values. In some embodiments, these increases in yeast biomass are accomplished without the production of detectable amounts of ethanol. In some embodiments, these increases in yeast biomass are accomplished with production of ethanol that results in a concentration of no more than 0.01, 0.05, 0.1, 0.15, 0.2, 0.25, 0.3, 0.35, 0.4, 0.45, or 0.5 g/L in the propagation medium.

### D. Propagation Conditions

The conditions at which the yeast are propagated are chosen so as to ensure efficient growth of yeast biomass and enzymatic hydrolysis of cellulosic material at rates conducive to efficient yeast biomass growth without inducing production of substantial amounts of ethanol. Important parameters include the temperature, pH, and aeration rate.

The temperature of the propagation medium is chosen so as to allow for simultaneous enzymatic hydrolysis of cellulosic material and yeast biomass production. In some embodiments, the temperature is in a range from 20 to 40° C., from 25 to 37° C., or from 30 to 32° C. In some embodiments, the temperature is at least about, at most about, or is about 20, 21, 22, 23, 24, 25, 26, 27, 28, 29, 30, 31, 32, 33, 34, 35, 36, 37, 38, 39, or 40° C. or is between any two of these values.

The propagation medium can be incubated at the chosen temperature for sufficient time to allow for a predetermined amount of yeast biomass production. In some embodiments, the propagation medium is incubated for 12 to 24 hours. In some embodiments, the propagation medium is incubated for about 10, 11, 12, 13, 14, 15, 16, 17, 18, 19, 20, 21, 22, 23, 24, 25, 26, 27, 28, 29, 30, 35, 40, 45, 50, 55, 60, 65, 70, or 72 hours or for an amount of time in a range between any two of these values.

The pH of the propagation medium affects enzymatic activity and yeast growth. In some embodiments, the pH of the propagation medium is in a range from about 4.0 to 6.0, from about 4.5 to 5.5, or from about 5.0 to 5.2. In some embodiments, the pH is at least about, at most about, or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, or 6, or is between any two of these values.

Aeration of the propagation medium ensures that sufficient oxygen is available to the growing yeast to support respiratory growth and avoid anaerobic fermentation. Aeration can be provided by any well-known apparatus such as an air sparging system. In some embodiments, the propagation medium is aerated at a rate of between 0.5 and 1.5 volumes of air per volume of medium per minute (vvm). In some embodiments, the aeration rate is about 0.5, 0.6, 0.7, 0.8, 0.9, 1.0, 1.1, 1.2, 1.3, 1.4, or 1.5, or between any two of these values.

The propagation medium additionally includes a nutrient source to support growth of yeast biomass. The nutrient source can include, for example, yeast extract, urea, diammonium phosphate, magnesium, zinc sulfate, and the like.

### E. Propagation System

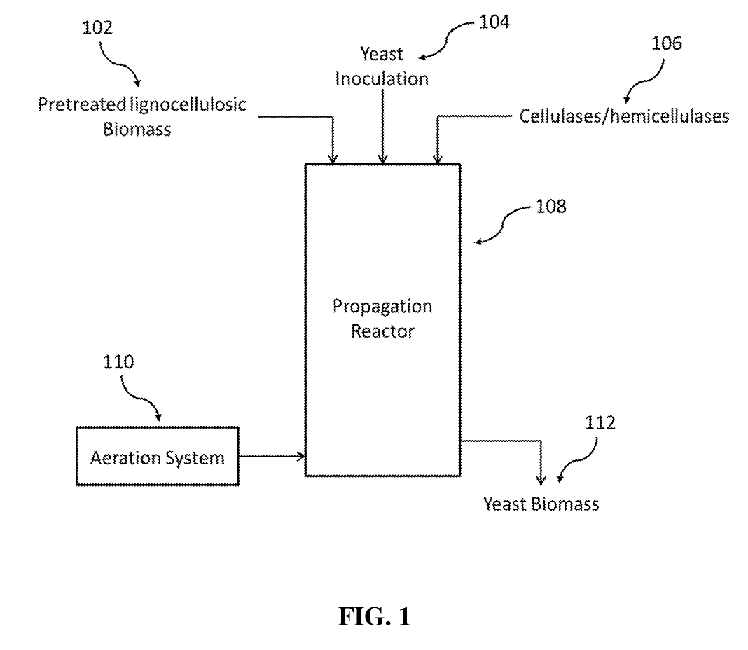

An embodiment of a propagation system that may be used in carrying out the methods described herein is illustrated in FIG. 1. As shown in FIG. 1, a pretreated lignocellulosic biomass 102 is added to a propagation reactor 108, which may be accomplished via a conveyor. Propagation reactors are well-known and can include stirred tanks as well as other reactors. The pretreated lignocellulosic biomass 102 acts as a carbon source for yeast biomass growth in the propagation reactor 108. Other cellulosic materials that do not include lignin can also be used as a carbon source.

In addition to the pretreated lignocellulosic biomass 102, an initial yeast inoculation 104 and a dosage of cellulases and/or hemicellulases (which may be an “enzyme cocktail”) 306 are added. Optionally, one or more enzymes may be added at a later stage after a lag phase of yeast growth has occurred. For example, a measurement system (not shown) can be used for adding the enzyme cocktail 106 to the reactor 108. The sugar (e.g., glucose) that is generated as the enzymes break down the starch functions as part of, or all of, a carbon source to propagate the yeast and produce yeast biomass. Although not shown in FIG. 1, a nutrient source can be added to reactor 108, as well as any optional agents that are well known for use in a yeast propagation medium.

The yeast inoculation 104 is allowed to propagate in the reactor 108 for a set period of time under high oxygen concentrations. Oxygen can be provided by an aeration system 110 which may sparge air through the reactor 308 and may also include mechanical agitation. After propagation, the resulting yeast biomass 112 can be collected and delivered to downstream applications.

### F. Example

A system similar to that shown in FIG. 1 was used in an experiment to study the use of unbleached kraft pulp treated by enzymes as a carbon source for propagating yeast, Saccharomyces cerevisiae. In this example, the active dry form of S. cerevisiae strain (ADY), C6 Fuel® from Lallemand was used. The growth medium for propagation of the yeast had unbleached kraft pulp as the sole carbon source at 30 g (dry)/L in combination with urea at 0.24 g/L, yeast extract at 1 g/L, and magnesium sulfate at 0.5 g/L.

One liter of unbleached kraft pulp at 3% solids was added to a 2 L Sartorius Bioreactor. To this pulp slurry, the required amounts of urea, yeast extract, and magnesium sulfate to attain the concentrations for growth medium given above were added and mixed well. The pH of the mixture was adjusted to 5.5 using 10% v/v sulfuric acid. The temperature of the reactor was set to 32° C. and maintained at this temperature for the entire length of the study (24 hours). To this growth media, C6 Fuel® active dry yeast (ADY) was added at 0.2 g/L. The reactor was agitated at 450 rpm, and airflow was adjusted to 1.0 standard liters per minute (SLPM), (1 volume of air per volume of medium per minute, vvm), to ensure adequate aeration. The cellulase cocktail Cellic CTec3 was added at 4.5%/g dry pulp solids. Two such reactors were set up—one with yeast and the other without yeast inoculation. The reactor without yeast inoculation was set up to see the amount of glucose produced by the enzyme under similar conditions. Samples were withdrawn at 24 hours and analyzed for glucose, xylose, ethanol and suspended solids. The results, summarized in Table 1 below, showed that all the glucose produced was consumed by the yeast (Table 1). Ethanol production was negligible. Based on the dry weight measurements, the yield of yeast biomass observed was 0.48 g (on dry basis) per g of glucose consumed. The yeast increased in mass 32 fold to a concentration of 6.4 g/L.

TABLE 1
Concentrations of glucose, xylose, and ethanol during propagation of yeast on
unbleached kraft pulp in the presence of cellulase enzyme. The suspended solids
measured at the end of propagation were to account for yeast biomass yield.

| | Glucose (% w/v) | | Xylose (% w/v) | | Ethanol (% w/v) | | Suspended solids (%) | |
|---|---|---|---|---|---|---|---|---|
| | With | Without | With | Without | With | Without | With | Without |
| Time (h) | Yeast | Yeast | Yeast | Yeast | Yeast | Yeast | Yeast | Yeast |
| 0 | 0.06 | 0.05 | 0.03 | 0.01 | 0.00 | 0.00 | — | — |
| 24 | 0.00 | 1.32 | 0.12 | 0.16 | 0.02 | 0.00 | 1.75 | 1.11 |

The above specification and examples provide a complete description of the implementation and structure of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary step or structure, and/or may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A method of propagating yeast comprising:
(a) combining:
(i) a propagation medium comprising a nutrient source;
(ii) a carbon source comprising a cellulosic material;
(iii) one or more enzymes that can convert at least a portion of the cellulosic material into one or more monosaccharides; and
(iv) a first cell mass of yeast;
(b) enzymatically converting at least a portion of the cellulosic material in the propagation medium into one or more monosaccharides; and
(c) incubating the propagation medium for sufficient time period to form a second cell mass of yeast that has a higher dry cell weight than the first cell mass of yeast;
wherein no more than 0.1 g/L of ethanol is produced in the propagation medium by the yeast during step (c).

2. The method of claim 1, wherein substantially no ethanol is produced by the yeast during step (c).

3. The method of claim 1, wherein the first cell mass of yeast has a dry cell weight of about 0.05 to 0.50 g per liter of propagation medium.

4. The method of claim 1, wherein the second cell mass of yeast has a dry cell weight of approximately 5 to 15 g per liter of propagation medium.

5. The method of claim 1, wherein the second cell mass of yeast has a dry cell weight approximately 25 to 100 times that of the first cell mass of yeast.

6. The method of claim 1, wherein the time period of step (c) is in a range from 12 to 24 hours.

7. The method of claim 1, wherein during step (b) and step (c) the propagation medium is maintained at a temperature in the range from 25 to 37° C.

8. The method of claim 1, wherein step (b) and step (c) occur simultaneously for at least a period of time.

9. The method of claim 1, wherein the propagation medium has a pH in a range from about 4 to about 6.

10. The method of claim 1, wherein the cellulosic material comprises lignocellulosic material.

11. The method of claim 1, wherein the one or more enzymes comprise one or more enzymes selected from the group of enzymes consisting of: an endoglucanase, a cellobiohydrolase, a xylanase, and a beta-glucosidase.

12. The method of claim 1, wherein the one or more enzymes are present in the propagation medium at a total enzyme concentration of about 0.02 to 0.7 grams of enzyme per gram of cellulosic material in the propagation medium.

13. The method of claim 1, wherein the one or more enzymes are not produced by the yeast in the first cell mass of yeast.

14. The method of claim 1, wherein the cellulosic material comprises cellulose derived from pulp generated by the Kraft or sulfite pulping process.

15. The method of claim 1, wherein the first cell mass of yeast comprises Saccharomyces cerevisiae.
